(19) United States
(12) Patent Application Publication
(10) Pub. No.: US 2003/0008012 A1
(43) Pub. Date: Jan. 9, 2003

(54) Composition for rectal delivery of an oxazolidinone antibacterial drug

(76) Inventors: Lorraine E. Pena, Kalamazoo, MI (US); Vincent E. McCurdy, Portage, MI (US); Carol S. Clark, Granger, IN (US)

Correspondence Address: Pharmacia & Upjohn Company, Patent Department, 800 N. Lindbergh Boulevard - 04E, St. Louis, MO, 63167, US

(21) Appl. No.: 10/072,493

(22) Filed: Feb. 5, 2002

Related U.S. Application Data
(60) Provisional application No. 60/266,528, filed on Feb. 5, 2001.
(60) Provisional application No. 60/285,260, filed on Apr. 20, 2001.

(51) Int. Cl. A61K 31/541; A61K 31/5377; A61K 9/14

(52) U.S. Cl. 424/489; 514/227.8; 514/235.5

(57) ABSTRACT

There is provided a pharmaceutical composition suitable for rectal administration, the composition comprising at least one oxazolidinone antibacterial drug, for example linezolid, in a concentration effective for treatment and/or prophylaxis of a gram-positive bacterial infection, the at least one oxazolidinone being in particulate form having a particle size of about 0.5 μm to about 150 μm, dispersed in a carrier in which the oxazolidinone is poorly soluble. The composition is, for example, a suppository, an enema, a microenema or a rectal capsule.

### FIELD OF THE INVENTION

The present invention relates to a pharmaceutical composition useful for rectal application for treatment or prevention of infective disease. In particular, the present invention relates to a rectal formulation of an oxazolidinone antibacterial drug that can be used for treatment or prevention of infection by a gram-positive bacterial agent. The field of the present invention also includes therapeutic or prophylactic use of such a formulation, and use of such a formulation in preparation of a medicament.

### BACKGROUND OF THE INVENTION

Numerous oxazolidinone compounds have been reported having therapeutically and/or prophylactically useful antibiotic, in particular antibacterial, effect. Among such compounds are those illustratively disclosed in the following patents, each of which is individually incorporated herein by reference.

U.S. Pat. No. 5,164,510 to Brickner.

U.S. Pat. No. 5,231,188 to Brickner.

U.S. Pat. No. 5,565,571 to Barbachyn & Brickner.

U.S. Pat. No. 5,627,181 to Riedl et al.

U.S. Pat. No. 5,652,238 to Barbachyn et al.

U.S. Pat. No. 5,688,792 to Barbachyn et al.

U.S. Pat. No. 5,698,574 to Riedl et al.

U.S. Pat. No. 6,069,145 to Betts.

Compounds disclosed in above-cited U.S. Pat. No. 5,688,792 include for example the compound (S)-N-[[3-[3-fluoro-4-(4-morpholinyl)phenyl]-2-oxo-5-oxazolidinyl]methyl]acetamide, also referred to herein as linezolid. Linezolid has the structure shown in formula (I):

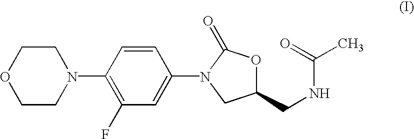

and is in commercial use as a medicament under the trademark Zyvox® of Pharmacia Corporation. Linezolid exhibits strong antibacterial activity against gram-positive organisms including those of the following genera: Staphylococcus (e.g., Staphylococcus aureus, Staphylococcus epidermidis), Streptococcus (e.g., Streptococcus viridans, Streptococcus pneumoniae), Enterococcus, Bacillus, Corynebacterium, Chlamydia and Neisseria. Many such gram-positive organisms have developed significant levels of resistance to other antibiotics.

Above-cited U.S. Pat. No. 5,688,792 discloses that the subject antibiotic oxazolidinone compounds, including linezolid, can be administered by either parenteral, oral or topical administration and can be formulated as solid dosage forms including powders, tablets, dispersible granules, capsules, cachets and suppositories, or as liquid dosage forms including solutions, suspensions and emulsions. However, U.S. Pat. No. 5,688,792 does not disclose or suggest any formulation comprising an oxazolidinone antibacterial drug adapted for systemic delivery by rectal administration.

It is well known that, although parenteral and oral routes of administration may be excellent for systemic delivery of drugs to many subjects, these routes may be less suitable for particular classes of subject. For example, some subjects such as small children, small adults, and elderly individuals have problems in swallowing a medication, or are otherwise incompliant with attempts at oral administration. Parenteral administration, in particular injection, likewise has disadvantages, for example in a requirement for administration by trained personnel and in a fear or sensation of pain that can be associated with such administration. Consequently, the rectal route would be advantageous in some instances for administration of an oxazolidinone antibacterial drug, if a suitable and effective formulation for such administration could be developed.

### SUMMARY OF THE INVENTION

The present invention arises in part from a finding that selection of a formulation having particular characteristics as defined hereinbelow is critical to providing effective systemic delivery of an oxazolidinone antibacterial drug by the rectal route of administration.

The invention provides a pharmaceutical composition useful for treatment and/or prophylaxis of a gram-positive bacterial infection in a subject, the composition comprising at least one oxazolidinone antibacterial drug in a solid particulate form having a volume median diameter of about 0.5 μm to about 150 μm dispersed in a pharmaceutically acceptable carrier in which the at least one oxazolidinone is poorly soluble, wherein the composition is adapted for rectal administration. The composition, optionally, further comprises at least one pharmaceutically acceptable excipient.

In one embodiment of the invention, the pharmaceutically acceptable carrier is liquid, such that the composition is adapted as a liquid dosage form for rectal administration, for example as an enema. In an alternative embodiment, the carrier is solid or semi-solid, such that the composition is adapted as a solid dosage form for rectal administration, for example as a suppository.

Rectal formulations according to the invention have advantages or benefits over known rectal formulations, including, but not limited to those presented below. In one common type of known rectal formulation, active agents are present in solution, either as an enema solution or emulsion, or contained within a suppository.

It is generally understood that partitioning of the active agent into the rectal membrane is more readily achieved when the active agent is administered in solution than it is when administered in particulate form. Surprisingly, formulations of the present invention, where an active agent is present in particulate form, exhibit high systemic bioavailability of the active agent following rectal administration, even when solubility of the active agent in the carrier is low.

The presence of the active agent in particulate form in the carrier rather than dissolved in the carrier, in the formulations of the present invention, allows for a smaller volume of composition to be administered for a given dose; because, active agent loading is not limited by solubility in the carrier. This makes the administration more practical and convenient to the subject. This is especially important where the maximum tolerable volume of administration is small, as for example where the subject is an infant or neonate.

Due to the fact that an active agent in the composition of the present invention, an oxazolidinone, is dispersed in particulate form in the carrier rather than dissolved in the carrier, the chemical stability of a composition according to the invention is typically better than for a composition where the drug is dissolved in the carrier. For example, certain drugs that exhibit chemical instability in solution are less prone to such instability when dispersed in a carrier in which they are poorly soluble or insoluble.

The absorption rate of the oxazolidinone antibacterial drug can be modified by varying the particle size of the oxazolidinone in a composition of the invention. This is not an option in a composition where an active agent is dissolved in the carrier.

Since the drug is not dissolved in the carrier, a lipophilic carrier can be used that would otherwise have been precluded due to the low solubility of oxazolidinone antibacterial drugs in such a carrier. Indeed, in a presently preferred embodiment of the invention the carrier is lipophilic. Use of a lipophilic carrier affords additional advantages or benefits, including without limitation those presented below.

The absorption rate of the active agent in the present composition can be modified by using lipophilic excipients with different physical and chemical properties.

Because a lipophilic carrier does not absorb water to any significant extent, the physical and chemical stability of a composition of the invention can be better than that of a composition having a hydrophilic carrier that absorbs water.

When the composition is formulated as a suppository, insertion of the suppository in the rectum is less unpleasant due to the softer consistency and lubricating effect of a solid lipophilic carrier in comparison with a hydrophilic solid carrier.

Discomfort associated with hydration of a hydrophilic carrier following insertion in the rectum is avoided.

A manufacturing process for a composition having a lipophilic carrier is convenient since there is no need to use additional mixing time, elevated temperature or increased agitation in order to dissolve the at least one oxazolidinone used in the present composition. If a solid dosage form is required, manufacturing is facilitated by low melting points of lipophilic carriers.

A composition of the invention that comprises a lipophilic carrier can be formulated as suppositories that melt at body temperature and consequently are able to release the at least one oxazolidinone without dissolution of the suppository. This is in contrast to hydrophilic suppositories, which normally are dependent on dissolution to release the at least one oxazolidinone.

Other features and benefits of the invention will be obvious from the following detailed description.

### DETAILED DESCRIPTION OF THE INVENTION

As indicated above, the invention provides a pharmaceutical composition suitable for rectal administration to treat and/or prevent a gram-positive bacterial infection. The composition comprises at least one oxazolidinone antibacterial drug in particulate form, dispersed in a pharmaceutically acceptable carrier in which the oxazolidinone is poorly soluble. The carrier is preferably lipophilic. The total concentration of oxazolidinone antibacterial drug in the composition is preferably an antimicrobially effective concentration for rectal administration to and treatment of or prophylaxis of a gram-positive bacterial infection of a subject. The composition preferably further comprises at least one pharmaceutically acceptable excipient.

In a preferred embodiment, the oxazolidinone antibacterial drug is a compound of formula (II)

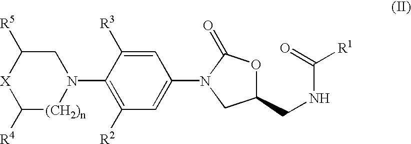

wherein:

R¹ is selected from (a) H, (b) C₁₋₈ alkyl optionally substituted with one or more of F, Cl, OH, C₁₋₈ alkoxy, C₁₋₈ acyloxy or benzoxy groups, and including C₃₋₆ cycloalkyl, (c) amino, (d) mono- and di(C₁₋₈ alkyl)amino and (e) C₁₋₈ alkoxy groups;

R² and R³ are independently selected from H, F and Cl groups;

R⁴ is H or CH₃;

R⁵ is selected from H, CH₃, CN, CO₂R¹ and (CH₂)ₘR⁶ groups, where R¹ is as defined above, R⁶ is selected from H, OH, OR¹, OCOR¹, NHCOR¹, amino, mono- and di(C₁₋₈ alkyl)amino groups and m is 1 or 2;

n is 0, 1 or 2; and

X is O, S, SO, SO₂, SNR⁷ or S(O)NR⁷ where R⁷ is selected from H, C₁₋₄ alkyl (optionally substituted with one or more F, Cl, OH, C₁₋₈ alkoxy, amino, C₁₋₈ mono- or di(C₁₋₈ alkyl)amino groups), and p-toluenesulfonyl groups;

or a pharmaceutically acceptable salt thereof.

A particularly preferred embodiment of the oxazolidinone antibacterial drug is a compound of formula (II), wherein R¹ is CH₃; R² and R³ are independently selected from H and F but at least one of R² and R³ is F; R⁴ and R⁵ are each H; n is 1; and X is O, S or SO₂. In another preferred embodiment, the oxazolidinone antibacterial drug is selected from the group consisting of: linezolid, eperezolid, N-((5S)-3-(3-fluoro-4-(4-(2-fluoroethyl)-3-oxopiperazin- 1-yl)phenyl)-2-oxooxazolidin-5-ylmethyl)acetamide, (S)-N-[[3-[5-(3-pyridyl)thiophen-2-yl]-2-oxo-5-oxazolidinyl]methyl]acetamide, (S)-N-[[3-[5-(4-pyridyl)pyrid-2-yl]-2-oxo-5-oxazolidinyl]methyl]acetamide hydrochloride and N-[[(5S)-3-[4-(1,1-dioxido-4-thiomorpholinyl)-3,5-difluorophenyl]-2-oxo-5-oxazolidinyl]methyl]acetamide. An especially preferred oxazolidinone antibacterial drug is linezolid. Another especially preferred oxazolidinone antibacterial drug is N-[[(5S)-3-[4-(1,1-dioxido-4-thiomorpholinyl)-3,5-difluorophenyl]-2-oxo-5-oxazolidinyl]methyl]acetamide.

The invention is illustrated herein with particular reference to linezolid. However, it will be understood that it is contemplated that any other oxazolidinone antibacterial compound, including any such compound of formula (II), as described above, can, be substituted in whole or in part for linezolid. In some cases, it will be necessary to make appropriate adjustment in concentration and dosage ranges to account for properties of the particular type of oxazolidinone used in the compositions and methods of the present invention, as described herein.

Oxazolidinone compounds used in compositions of the invention can be prepared by a process known per se, in the case of linezolid and eperezolid, for example, by processes described in the following patents, each of which is individually incorporated herein by reference.

Above-cited U.S. Pat. No. 5,688,792.

U.S. Pat. No. 5,837,870 to Barbachyn et al.

International Patent Publication No. WO 99/24393.

Other oxazolidinone antibacterial drugs can be prepared by processes known per se, including processes set forth in patent publications disclosing such drugs.

Although compositions of the present invention are contemplated to be especially useful when administered rectally as described herein, such compositions can also have utility as antibacterial medicaments by other routes of administration, for example by vaginal or urethral administration. When administered vaginally or urethrally, such compositions can provide either local or systemic antibacterial effect or both; however, where systemic effect is desired, rectal administration is the preferred route.

The term “poorly soluble” herein, in relation to solubility of the at least one oxazolidinone in the carrier, means having a solubility of less than about 50 mg/ml, preferably less than about 25 mg/ml, more preferably less than about 10 mg/ml.

The term “in particulate form” herein means that an active agent, such as the at least one oxazolidinone, is not completely dissolved (i.e., molecularly dispersed) in the solid or liquid carrier but is at least to some extent present as multimolecular particles in the carrier. The particles have a particle size of not more than about 150 μm, preferably not more than about 20 μm. Small particle sizes are generally preferred in order to avoid sedimentation, to minimize rectal irritation and to enhance dissolution rate. The minimum particle size is not critical, but should not be so small as to cause problems in manufacture. Particle size as small as about 0.5 μm is satisfactory. If the particle size of the active agent used in preparing a composition of the invention is greater than about 20 μm, it can be reduced by any conventional means, for example by milling using a pulverizing rotary mill or air jet micronizer.

The term “particle size” herein refers to volume median diameter, as measured by any suitable technique, preferably using a laser diffraction instrument (e.g., a Sympatec Helos). It should be noted that for particles having an irregular shape, such as acicular particles, the volume median diameter as measured by laser diffraction will be significantly smaller than the average of the longest dimension of the particle population.

The carrier used in the present invention is liquid, semisolid, or solid at room temperature, or a mixture of any or all of these states. A lipophilic carrier useful herein comprises one or more pharmaceutically acceptable excipients, and is essentially insoluble in water. Where the lipophilic carrier is solid at room temperature it must melt or soften at body temperature in order to allow release of the at least one oxazolidinone dispersed therein. A preferred lipophilic carrier comprises one or more mono-, di- or triglycerides of one or more saturated, unsaturated or polyunsaturated fatty acids. An especially preferred solid lipophilic carrier is a hard fat or mixture of hard fats.

When a lipophilic carrier that is solid at room temperature is used, it preferably has a flow point of about 25° C. to about 40° C., more preferably about 30° C. to about 37° C. The flow point can be visually determined by heating a sample of the carrier from 25° C. at a rate of 2° C./minute and observing the temperature at which rapid flow of the sample first occurs. This measurement is conveniently carried out using a microscope equipped with a video camera having on-screen digital monitoring of the temperature. It is common for solid lipophilic carriers, such as hard fat, to undergo a polymorphic transition to the most stable form during storage, in the case of hard fat conversion to the β-polymorphic form. The flow points described above refer to flow points following completion of such polymorphic transition. Using conventional x-ray diffraction techniques, the polymorphic transition to the most stable form can be monitored from the time of the initial manufacture until no further changes in the diffraction pattern over a period of about a month are evident.

It is desirable that a large part of the total concentration of oxazolidinone in the composition is absorbed into systemic circulation following rectal administration. Preferably, bioavailability following rectal administration is greater than about 25%, more preferably greater than about 50%, for example greater than about 80%.

The amount of oxazolidinone that is incorporated into the composition can be varied depending on the dose that is desired. The percent by weight of active agent incorporated into the composition (i.e., the active agent or drug loading) preferably ranges from about 0.1% to about 50%, more preferably from about 1% to about 25%. A liquid composition of the invention, such as an enema, can generally be administered comfortably in significantly greater volume than a solid composition of the invention, such as a suppository. In a solid composition, the drug loading, as a percent by weight of active agent incorporated into the composition, is preferably about 1% to about 50%, more preferably about 3% to about 25%.

The present invention also provides a method of treating and/or preventing both gram-positive and gram-negative bacterial infections, the method comprising rectal administration in co-therapy of one or more oxazolidinone antibacterial drugs and one or more antibacterial drugs other than oxazolidinones effective against gram-negative organisms. Co-therapy herein includes, without restriction, coformulation of the oxazolidinone and non-oxazolidinone drugs in a single composition. Thus, the present invention also provides a pharmaceutical composition suitable for rectal administration, the composition comprising as active agents (a) one or more oxazolidinone antibacterial drugs in an amount effective for treatment and/or prophylaxis of a gram-positive bacterial infection, and (b) one or more antibacterial drugs other than oxazolidinones in an amount effective for treatment and/or prophylaxis of a gram-negative bacterial infection.

Any gram-negative effective antibiotic that is sufficiently absorbed when administered rectally can be used in such co-therapy or coformulation with one or more oxazolidinone antibiotics in accordance with this embodiment of the invention. Suitable gram-negative effective antibiotics can be selected, without limitation, from aminoglycosides, cephalosporins, diaminopyridines, fluroquinolones, sulfonamides and tetracyclines. Among particular antibiotics of these and other classes, each of the following may illustratively be useful as a gram-negative effective antibiotic: amikacin, ampicillin, azithromycin, aztreonam, carbapenam, cefazolin, ceftazidime, cefixime, ceftriaxone, cefoperazone, cefotaxime, ceftizoxime, cefuroxime, chloramphenicol, ciprofloxacin, clindamycin, colistin, domeclocycline, doxycycline, erythromycin, gentamicin, imipenem, levofloxacin, mafenide, methacycline, metronidazole, minocycline, neomycin, norfloxacin, ofloxacin, oxytetracycline, piperacillin, polymyxin B, pyrimethamine, silver sulfadiazine, sulbactam, sulfacetamide, sulfisoxazole, tetracycline, tobramycin, trimethoprim and quinolone. Presently preferred gram-negative effective antibiotics are azithromycin, carbapenam, ceftazidime, ceftriaxone, cefuroxime, ciprofloxacin, erythromycin, gentamicin, imipenem, metronidazole and quinolone.

Preferably, such co-therapy or coformulation is with an oxazolidinone-containing composition as hereinabove described, i.e., a composition adapted for rectal administration, comprising at least one oxazolidinone antibacterial drug, dispersed in particulate form in a carrier in which the at least one oxazolidinone drug is poorly soluble, preferably a lipophilic carrier, and further comprising one or more gram-negative effective antibacterial drugs other than oxazolidinones. Such a coformulated composition represents a further embodiment of the present invention. The non-oxazolidinone component can be, like the oxazolidinone component, dispersed in particulate form in the carrier, or it can be dissolved therein.

In a particularly preferred embodiment, the present invention is a suppository, formulated as illustrated below. However, the invention can alternatively be formulated into other dosage forms such as an enema, a microenema or a rectal capsule as will be understood by a person skilled in the art. For an enema or microenema it is important to use a carrier which is liquid at room temperature and has a suitable viscosity when the active agent is dispersed therein. An example of a suitable liquid lipophilic carrier is caprylic/capric triglyceride (e.g., Miglyol™ 810 and Miglyol™ 812). It may be necessary to control particle size of the active agent and to include additives in order to avoid segregation as is known in the art. For rectal capsules, the active agent is dispersed in a liquid carrier, preferably a liquid lipophilic carrier (which may or may not be solid at room temperature but is held during addition of the at least one oxazolidinone at a temperature above its melting point) and filled into capsules, for example hard or soft gelatin capsules, as will be understood by a person skilled in the art of capsule filling.

The total weight of a suppository of the invention varies according to the total concentration (i.e., the desired dose) of oxazolidinone and any other active agents (e.g., gram-negative antibacterial agents) and “ease of use” characteristics such as size and shape of the resulting suppository, and is therefore not critical. Generally, lower amounts of active ingredient may be accommodated by a smaller size of suppository, and higher amounts of active ingredient will require a larger size of suppository. Manufacturing properties, such as the viscosity of the dispersion of active agent in the carrier when the carrier is in a molten state during processing, will also determine the minimum amount of suppository carrier that is needed to disperse, mold and package a suppository having a given amount of active agent. Such a parameter is not critical to the present invention, and may be determined in the course of routine optimization of the manufacturing process. Typical suppositories have a weight of about 0.1 to about 10 g, preferably about 0.2 to about 5 g, and most preferably about 0.3 to about 3 g. Small suppositories, for example about 0.2 to about 1 g in weight, are especially suitable for administration to neonates, infants and small children, while larger suppositories, for example about 1 to about 5 g in weight, are more suitable for administration to adult subjects.

In a particularly preferred embodiment, a hard fat suppository base is used as the lipophilic carrier. Examples of useful hard fat suppository bases are manufactured by Condea Vista Company, Cranford, N.J. under the Witepsol™ trademark, e.g., the Witepsol™ H-series and W-series of suppository bases, and by Stepan Company, Northfield, Ill. under the Wecobee™ trademark.

Further useful carriers are those manufactured by Gattefossé Etablissements, Saint Priest, France under the Suppocire™ trademark. The Witepsol™ bases are described by their manufacturer as being “glyceride esters of saturated C₁₂-C₁₈ fatty acids.” The Wecobee™ bases are described by their manufacturer as being “a triglyceride derived from vegetable oil.” The Suppocire™ bases are described by their manufacturer as hydrogenated palm kernel glycerides and hydrogenated palm glycerides.

The most preferred hard fat suppository base is a mixture of glyceride esters of vegetable C₁₂-C₁₈ saturated fatty acids. The majority of the glyceride esters are preferably triglycerides. These most preferred suppository bases have the following characteristics in the absence of active agent:

| | |
|---|---|
| Open-tube melting point: | about 31-37° C. (α-polymorphic form); |
| Solidification point: | about 25-35° C. (α-polymorphic form); |
| Hydroxyl value: | not more than about 50 mg KOH/g; |
| Saponification value: | about 220-260 mg KOH/g; |
| Diglycerides: | not more than about 35% by weight; |
| Monoglycerides: | not more than about 5% by weight. |

The vegetable source is preferably coconut and palm kernel oils.

An illustrative hard fat base is a mixture of triglyceride esters of coconut and palm kernel oil C₁₂-C₁₈ saturated fatty acids having the following characteristics in the absence of active agent:

| | |
|---|---|
| Open-tube melting point: | about 31-36° C. (α-polymorphic form); |
| Solidification point: | about 30-35° C. (α-polymorphic form); |
| Hydroxyl value: | not more than about 15 mg KOH/g; |
| Saponification value: | about 230-250 mg KOH/g; |
| Diglycerides: | not more than about 15% by weight; |
| Monoglycerides: | not more than about 1% by weight. |

All the above tests should be performed in accordance with standardized procedures, e.g., those of United States Pharmacopeia or European Pharmacopoeia.

The carriers for use in accordance with the invention can be produced by any conventional means. One such means involves blending C₁₂-C₁₈ saturated fatty acids, preferably derived from coconut and palm kernel oils, followed by esterifying the mixture with glycerol. Routine variations in the blend of saturated fatty acids and in the esterification conditions will enable the production of suppository carriers having the desired properties. Examples of commercially available carriers which meet the illustrative specification above are Witepsol™ H-15 and Witepsol™ H-32. An example of a composition of a linezolid rectal suppository of the present invention, produced using Witepsol™ H-32 is presented in Table 1, below.

TABLE 1

| Component | Amount per suppository |
|---|---|
| Linezolid (milled) | 104 mg |
| Witepsol ™ H-32 (hard fat) | 616 mg |

The suppositories of the present invention preferably further contain additional pharmaceutically acceptable excipients, such as stabilizers (e.g., antioxidants and other types of preservatives), polymorphic transition accelerators (e.g., tristearin), biocompatible polymers, surfactants, dispersants, water absorbents, glycerin and the like. The use of biocompatible polymers, surfactants and water absorbents in a suppository formulation is described in U.S. Pat. No. 4,765,978 to Abidi & Sequeira, the disclosure of which is incorporated herein by reference. The concentration of these additional excipients can vary according to the particular excipient used and the desired result sought. Selection of excipients and optimization of the concentration thereof are well within the ability of the skilled artisan.

A rectal suppository of the present invention can be administered at a dosage, frequency and duration sufficient to treat the infection of the subject. The dosage regimen can vary depending on the particular oxazolidinone antibacterial drug selected, the type, locus and severity of the bacterial infection, the infective organism and the weight and age of the subject. For linezolid an illustrative treatment of an adult can comprise twice daily administration of one suppository containing about 400 to about 600 mg of linezolid, for a period of about 10 to about 28 days. An illustrative treatment of a neonate, an infant or a child can comprise administration of one suppository containing about 8 to about 12 mg of linezolid per kg body weight of the subject, 2 to 3 times daily for a period of about 10 to about 28 days.

Suppositories according to the invention can be prepared by any conventional means, such as by hand casting or through the use of an automated “form-fill-seal” suppository machine. In general terms, suppository manufacture can be performed by a process including the following steps: (a) melting the carrier at an appropriately selected elevated temperature, (b) incorporating the active agent into the resulting molten carrier, (c) mixing to form a uniform molten dispersion, (d) filling the molten dispersion into a suppository mold, and (e) cooling the dispersion to form a solid suppository. If desired, the molten carrier can be filtered prior to drug addition, and the drug/carrier mixture can be homogenized prior to mold filling. The molten dispersion is maintained at the elevated temperature for filling. If hand filled, the molten dispersion is volumetrically filled into casting molds and allowed to solidify at or below room temperature. The finished suppositories can then be individually packaged into preformed foil pouches or wrapped. Alternatively, the suppository manufacture can be automated using a form-fill-seal machine. By this method of manufacture, an open foil shell is formed by the machine and the molten suppository carrier is volumetrically filled into the shell. The foil is then sealed and the filled shell is transferred to a cooling table or other similar device for solidification.

In accordance with the above disclosure, an especially preferred embodiment of the invention is a rectal suppository comprising about 3% to about 25% solid particulate linezolid having a particle size of about 20 μm or less, dispersed in a hard fat carrier. The suppository is solid at room temperature, and has a flow point of about 37° C. or less after reaching the β-polymorphic form.

### EXAMPLES

### Example 1:

### Enema

A suspension composition suitable as an enema containing 1% linezolid by weight was prepared by the following procedure.

1. 10.0 g polysorbate 80 (Tween™ 80), 474.97 g caprylic/capric triglyceride (Miglyol™ 812) and 5.03 g linezolid, milled to a particle size of 14 μm, were combined and mixed, using a propeller mixer.

2. 10.0 g colloidal silicon dioxide (Cab-O-Sil™) was added to the resulting mixture.

3. The mixture was then mixed for 10 minutes, using a propeller mixer to form a suspension.

4. The suspension was then homogenized in a Silverson homogenizer for 3 minutes.

### Example 2:

### Solution Suppository (Comparative Example)

Suppositories containing 0.8% linezolid by weight, in solution in a hydrophilic carrier, were prepared by the following procedure.

1. 99.2 g of polyethylene glycol 4000 (Carbowax™ 3350) was melted by heating to 70-71° C. in a jacketed beaker connected to a water bath. The polyethylene glycol was stirred by hand in order to facilitate melting.

2. 0.80 g unmilled linezolid was added to the resulting melted polyethylene glycol. The mixture was stirred by hand.

3. The linezolid was dissolved in the melted polyethylene glycol by high-speed homogenization for approximately 5 minutes, using a Silverson homogenizer.

4. The resulting solution of linezolid in molten polyethylene glycol was filled into suppository molds and allowed to cool at room temperature overnight.

5. The resulting solidified suppositories were removed from the mold.

The suppositories were smooth and white and had an average weight of 2.86 g. The average linezolid dosage amount per suppository was 20 mg.

One 20 mg linezolid suppository prepared as above was rectally administered to each of four beagle dogs, and the concentration of linezolid in blood plasma of the dogs was determined at different times after administration, as presented in Table 2.

TABLE 2

Linezolid concentration (μg/ml)

| Time (h) | Dog 1* | Dog 2* | Dog 3* | Dog 4* |
|---|---|---|---|---|
| 0.25 | 0.0476 | 0.0546 | 0.134 | 0.0807 |
| 0.5 | 0.132 | 0.136 | 0.305 | 0.204 |
| 0.75 | 0.168 | 0.189 | 0.340 | 0.291 |
| 1 | 0.209 | 0.197 | 0.388 | 0.394 |
| 1.5 | 0.267 | 0.179 | 0.423 | 0.532 |
| 2 | 0.354 | 0.238 | 0.351 | 0.548 |
| 2.5 | 0.417 | 0.171 | 0.331 | 0.550 |
| 3 | 0.364 | 0.146 | 0.317 | 0.556 |

* Body weights: Dog 1, 15.2 kg; Dog 2, 13.7 kg; Dog 3, 10.5 kg; Dog 4, 12.2 kg.

### Example 3:

### Particulate Dispersion Suppository

Suppositories containing 2.9% linezolid by weight, in particulate form dispersed in a lipophilic carrier, were prepared by the following procedure:

1. 97.123 g of hard fat (Witepsol™ H-32) was melted by heating to 40-42° C. in a jacketed beaker connected to a water bath. The hard fat was occasionally stirred by hand in order to facilitate melting.

2. 2.877 g linezolid, milled to a particle size of 14 μm, was added to the melted hard fat and mixed by stirring by hand.

3. The resulting linezolid hard fat mixture was then homogenized at high speed for 4-5 minutes, using a Silverson homogenizer.

4. The homogenized mixture of linezolid and molten hard fat was filled into suppository molds (approximately 0.35 g in each mold) and allowed to cool at room temperature overnight.

5. The resulting solidified suppositories were removed from the molds. The suppositories had an average weight of 322 mg.

### Example 4:

### Particulate Dispersion Suppository

Suppositories containing 14% linezolid by weight, in particulate form dispersed in a lipophilic carrier, were prepared by the following procedure.

1. 85.614 g of hard fat (Witepsol™ H-32) was melted by heating to 40-42° C. in a jacketed beaker connected to a water bath. The hard fat was occasionally stirred by hand in order to facilitate melting.

2. 14.386 g linezolid, milled to a particle size of 14 μm, was added to the melted hard fat. The mixture was stirred by hand.

3. The resulting linezolid hard fat mixture was then homogenized at high speed for 4-5 minutes, using a Silverson homogenizer.

4. The homogenized mixture of linezolid and molten hard fat was filled into suppository molds (approximately 0.7 g in each mold) and allowed to cool at room temperature overnight.

5. The resulting solidified suppositories were removed from the molds. The suppositories had an average weight of 720 mg.

### Example 5:

### Particulate Dispersion Suppository

Suppositories containing 24% linezolid by weight, in particulate form dispersed in a lipophilic carrier, were prepared by the following procedure.

1. 75.856 g of hard fat (Witepsol™ H-32) was melted by heating to 40-42° C. in a jacketed beaker connected to a water bath. The hard fat was occasionally stirred by hand in order to facilitate melting.

2. 24.144 g linezolid, milled to a particle size of 14 μm, was added to the melted hard fat. The mixture was stirred by hand.

3. The resulting linezolid hard fat mixture was then homogenized at high speed for 5 minutes, using a Silverson homogenizer.

4. The homogenized mixture of linezolid and molten hard fat was filled into suppository molds (approximately 2.5 g in each mold) and allowed to cool at room temperature overnight.

5. The resulting solidified suppositories were removed from the molds.

The average linezolid dosage amount per suppository was 600 mg.

One 600 mg linezolid suppository prepared as above was rectally administered to each of four beagle dogs and the concentration of linezolid in blood plasma of the dogs was determined at different times after administration, as presented in Table 3.

TABLE 3

Linezolid concentration (μg/ml)

| Time (h) | Dog 1* | Dog 2* | Dog 3* | Dog 4* |
|---|---|---|---|---|
| 0.25 | 1.11 | 0.77 | 0.444 | 1.1 |
| 0.5 | 2.25 | 1.24 | 1.11 | 1.72 |
| 0.75 | 3.84 | 1.94 | 1.68 | 1.93 |
| 1 | 5.01 | 2.61 | 2.54 | 2.64 |
| 1.5 | 6.26 | 3.49 | 3.46 | 4.26 |
| 2 | 7.23 | 3.57 | 4.84 | 4.81 |
| 2.5 | 7.98 | 5.01 | 5.51 | 6.06 |
| 3 | 8.48 | 4.73 | 6.57 | 7.48 |

* Body weights: Dog 1, 15.0 kg; Dog 2, 13.4 g; Dog 3, 10.1 kg; Dog 4, 12.5 kg.

Examples 3-5 illustrate the relatively high drug load that is possible with suppositories of the invention by comparison with those of comparative Example 2. The higher drug load permits higher drug dosages to be administered. Consequently, higher drug concentrations in blood plasma are possible by use of suppositories of the invention as illustrated by a comparison of Tables 2 and 3.

What is claimed is:

1. A pharmaceutical composition, comprising at least one oxazolidinone antibacterial drug in a solid particulate form dispersed in a pharmaceutically acceptable carrier in which the at least one oxazolidinone is poorly soluble, said composition being adapted for rectal administration.

2. The composition of claim 1 wherein the at least one oxazolidinone antibacterial drug is a compound of formula (I): 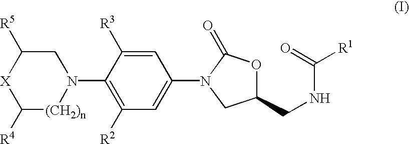
wherein:
R¹ is selected from (a) H, (b) C₁₋₈ alkyl optionally substituted with one or more F, Cl, OH, C₁₋₈ alkoxy, C₁₋₈ acyloxy or benzoxy groups, and including C₃₋₆ cycloalkyl, (c) amino, (d) mono- and di(C₁₋₈ alkyl)amino and (e) C₁₋₈ alkoxy groups;
R² and R³ are independently selected from H, F and Cl groups;
R⁴ is H or CH₃;
R⁵ is selected from H, CH₃, CN, CO₂R¹ and (CH₂)ₘR⁶ groups, where R¹ is as defined above, R⁶ is selected from H, OH, OR¹, OCOR¹, NHCOR¹, amino, mono- and di(C₁₋₈ alkyl)amino groups and m is 1 or 2;
n is 0, 1 or 2; and
X is O, S, SO, SO₂, SNR⁷ or S(O)NR⁷ where R⁷ is selected from H, C₁₋₄ alkyl (optionally substituted with one or more F, Cl, OH, C₁₋₈ alkoxy, amino, C₁₋₈ mono- or di(C₁₋₈ alkyl)amino groups), and p-toluenesulfonyl groups;
or a pharmaceutically acceptable salt thereof.

3. The composition of claim 1, wherein the solid particulate form of the at least one oxazolidinone has a volume median diameter of about 0.5 μm to about 150 μm.

4. The composition of claim 1 wherein the pharmaceutically acceptable carrier is lipophilic.

5. The composition of claim 4 wherein the lipophilic carrier is solid at room temperature.

6. The composition of claim 1 having a bioavailability of at least 25% of the total concentration of oxazolidinone in a dose of the composition administered to a subject.

7. The composition of claim 1, wherein the total concentration of oxazolidinone in the composition is sufficient to be effective for treatment and/or prophylaxis of a gram-positive bacterial infection in a subject when administered thereto.

8. The composition of claim 1 wherein the total concentration of oxazolidinone in the composition is about 0.1% to about 50% by weight.

9. The composition of claim 1 which is a dosage form selected from the group consisting of suppository, enema, microenema and rectal capsule.

10. The composition of claim 4 wherein the lipophilic carrier comprises a glyceride of fatty acids or a mixture of glycerides of fatty acids.

11. The composition of claim 10 wherein the lipophilic carrier comprises a hard fat.

12. The composition of claim 11 wherein the hard fat has a β-polymorphic form which has a flow point of about 25° C. to about 40° C.

13. The composition of claim 11 wherein the hard fat is a mixture of glyceride esters of vegetable C₁₂-C₁₈ saturated fatty acids containing more than about 50% triglyceride esters.

14. The composition of claim 13 wherein the hard fat has an open-tube melting point of about 31-36° C. in its α-polymorphic form; a solidification point of about 30-35° C. in its α-polymorphic form; a hydroxyl value of not more than about 15 mg KOH/g; a saponification value of about 230-250 mg KOH/g; diglyceride content not more than about 15% by weight; and monoglyceride content not more than about 1% by weight.

15. The composition of claim 4 which is solid and has a weight of about 0.1 g to about 10 g.

16. The composition of claim 1, wherein the at least one oxazolidinone antibacterial drug has a particle size of less than about 20 μm.

17. The composition of claim 1 wherein the at least one oxazolidinone antibacterial drug is linezolid.

18. The composition of claim 1 wherein the at least one oxazolidinone antibacterial drug is N-[[(5S)-3-[4-(1,1-dioxido-4-thiomorpholinyl)-3,5-difluorophenyl)-2-oxo-5-oxazolidinyl]methyl]acetamide.

19. The composition of claim 1, further comprising at least one antibacterial drug, other than an oxazolidinone, effective against gram-negative bacteria.

20. The composition of claim 19 wherein the at least one antibacterial drug effective against gram-negative bacteria is selected from the group consisting of: amikacin, ampicillin, azithromycin, aztreonam, carbapenam, cefazolin, ceftazidime, cefixime, ceftriaxone, cefoperazone, cefotaxime, ceftizoxime, cefuroxime, chloramphenicol, ciprofloxacin, clindamycin, colistin, domeclocycline, doxycycline, erythromycin, gentamicin, imipenem, levofloxacin, mafenide, methacycline, metronidazole, minocycline, neomycin, norfloxacin, ofloxacin, oxytetracycline, piperacillin, polymyxin B, pyrimethamine, silver sulfadiazine, sulbactam, sulfacetamide, sulfisoxazole, tetracycline, tobramycin, trimethoprim and quinolone.

21. A method of treatment or prevention of a gram-positive bacterial infection in a subject comprising:
(a) providing a pharmaceutical composition, comprising at least one oxazolidinone antibacterial drug in a solid particulate form dispersed in a pharmaceutically acceptable carrier in which the at least one oxazolidinone is poorly soluble, said composition being adapted for rectal administration; and
(b) rectally administering the pharmaceutical composition to the subject.

22. The method of claim 21, wherein the solid particulate form of the at least one oxazolidinone provided in step (a) has a volume median diameter of about 0.5 μm to about 150 μm.

23. The method of claim 21, wherein the at least one oxazolidinone antibacterial drug is a compound of formula (II): 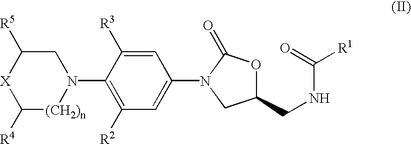
wherein:
R¹ is selected from (a) H, (b) C₁₋₈ alkyl optionally substituted with one or more F, Cl, OH, C₁₋₈ alkoxy, C₁₋₈ acyloxy or benzoxy groups, and including C₃₋₆ cycloalkyl, (c) amino, (d) mono- and di(C₁₋₈ alkyl)amino and (e) C₁₋₈ alkoxy groups;
R² and R³ are independently selected from H, F and Cl groups;
R⁴ is H or CH₃;
R⁵is selected from H, CH₃, CN, CO₂R¹ and (CH₂)ₘR⁶ groups, where R¹ is as defined above, R⁶ is selected from H, OH, OR¹, OCOR¹, NHCOR¹, amino, mono- and di(C₁₋₈ alkyl)amino groups and m is 1 or 2;
n is 0, 1 or 2; and
X is O, S, SO, SO₂, SNR⁷ or S(O)NR⁷ where R⁷ is selected from H, C₁₋₄ alkyl (optionally substituted with one or more F, Cl, OH, C₁₋₈ alkoxy, amino, C₁₋₈ mono- or di(C₁₋₈ alkyl)amino groups), and p-toluenesulfonyl groups;
or a pharmaceutically acceptable salt thereof.

24. The method of claim 21, wherein the total concentration of oxazolidinone in the pharmaceutical composition provided in step (a) is sufficient to be effective for treatment and/or prophylaxis of a gram-positive bacterial infection in the subject when administered thereto in step (b).

25. The method of claim 21, wherein the pharmaceutical composition further comprises at least one antibacterial drug effective against gram-negative bacteria.

26. The method of claim 25 wherein the at least one antibacterial drug effective against gram-negative bacteria is selected from the group consisting of amikacin, ampicillin, azithromycin, aztreonam, carbapenam, cefazolin, ceftazidime, cefixime, ceftriaxone, cefoperazone, cefotaxime, ceftizoxime, cefuroxime, chloramphenicol, ciprofloxacin, clindamycin, colistin, domeclocycline, doxycycline, erythromycin, gentamicin, imipenem, levofloxacin, mafenide, methacycline, metronidazole, minocycline, neomycin, norfloxacin, ofloxacin, oxytetracycline, piperacillin, polymyxin B, pyrimethamine, silver sulfadiazine, sulbactam, sulfacetamide, sulfisoxazole, tetracycline, tobramycin, trimethoprim and quinolone.

27. The method of claim 21 wherein the at least one oxazolidinone antibacterial drug is linezolid.

28. The method of claim 27, wherein the subject is an adult human and about 400 to about 600 mg of the linezolid is administered rectally twice daily to the subject for a period of about 10 to about 28 days.

29. The method of claim 27, wherein the subject is a human child and about 8 to about 12 mg linezolid per kg body weight is administered rectally 2 to 3 times daily for a period of about 10 to about 28 days.
